(19) United States
(12) Patent Application Publication
(10) Pub. No.: US 2011/0111451 A1
(43) Pub. Date: May 12, 2011

(54) Gross Hematuria Evaluator and Methods for Making and Using Same

(76) Inventor: Steven Y. Chung, Ottawa, IL (US)

(21) Appl. No.: 12/617,345

(22) Filed: Nov. 12, 2009

(51) Int. Cl. C12Q 1/04; C12M 1/34

(52) U.S. Cl. 435/34; 435/288.6

(57) ABSTRACT

A device for evaluating the blood content in a plurality of media may include a display having a plurality of columns, each column having a plurality of color-block rows, where the colors are created by diluting blood in the respective medium to obtain a color, and then substantially matching that color with a row in the column. Additionally or alternatively, the columns may represent blood diluted in the same sample, but observed at different times. The display may include gaps between columns to accept a catheter in which the sample to be analyzed is placed and a legend identifying the medium and blood dilution for each column and row, as well as the time of aging, if any, for a column.

### BACKGROUND OF THE INVENTION

1. Field of the Invention

The present invention is directed to a device for analyzing and evaluating the degree of hematuria or other internal bleeding in a patient and methods for making and using the device.

2. Description of the Related Art

Patients undergoing urological procedures or testing may experience internal bleeding, which may manifest as hematuria, or blood in the patients' urine. Myriad hematuria states (dilutions) may exist, and each state may dramatically affect treatment plans. In many cases, these patients may have catheters inserted in order to collect and analyze the urine in order to assess the state and severity of such bleeding. In addition, the catheter may have multiple ports, so as to allow for irrigation with saline or other solutions such as glycine, sorbitol, or sterile water, in order to treat the bleeding in patients with active bleeds. At times, a health care provider is able to view a patient's catheter first-hand and use that first-hand knowledge to make these assessments. At other times, however, the provider may not be present physically and, instead, must rely on the subjective assessment of another individual. In such cases, this subjective analysis may be inaccurate or not very helpful. Furthermore, even the slightest difference in color description may affect treatment plans.

In addition, patients often are monitored over time to determine their progress. The same individual that performed a first analysis may not be the same person to perform a later analysis, and the two individuals' subjective assessments may not provide a completely accurate picture to the health care provider. Similarly, the same individual may make both assessments, but may not remember what the earlier sample looked like in absolute terms or relative to the newer sample.

What are needed are a device and methods for making and using the device that overcome the drawbacks described above.

### BRIEF SUMMARY OF THE INVENTION

In one aspect of the invention, a device for evaluating blood content in a plurality of media, comprising: a plurality of columns, each column representing blood contents in different media; a plurality of color-filled boxes in each of the columns, wherein, for each column, each color correlates to an actual dilution of blood in one of the different media. The device further may comprise a gap between each of the plurality of columns, wherein the gap is at least as wide as tubing of a catheter containing a sample to be evaluated.

Each of the plurality of columns may have the same number of boxes, and the columns may be substantially aligned so that said boxes form rows across the columns. Boxes in each row may have substantially the same blood dilution percentage as the other boxes in that row. In addition, the device may include indicators or labels for each of the columns and each of the rows, forming a grid for selecting matching colors.

In another aspect of the invention, a method for making a device for evaluating blood content in a plurality of media may comprise: diluting a first percentage of blood in a first medium to obtain a first color; diluting a second percentage of blood in the first medium to obtain a second color; diluting a third percentage of blood in the first medium to obtain a third color; diluting a fourth percentage of blood in a second medium to obtain a fourth color; diluting a fifth percentage of blood in the second medium to obtain a fifth color; diluting a sixth percentage of blood in the second medium to obtain a sixth color; creating a first column having color-filled boxes filled with the first, second, and third colors; and creating a second column having color-filled boxes filled with the fourth, fifth, and sixth colors. The method also may comprise: diluting a seventh percentage of blood in a third medium to obtain a seventh color; diluting an eighth percentage of blood in the third medium to obtain an eighth color; diluting a ninth percentage of blood in the third medium to obtain a ninth color; and creating a third column having color-filled boxes filled with the seventh, eighth, and ninth colors. Alternatively, the method may comprise: aging the fourth percentage of blood in the second medium to obtain a seventh color; aging the fifth percentage of blood in the second medium to obtain an eighth color; aging the sixth percentage of blood in the second medium to obtain a ninth color, wherein the seventh, eighth, and ninth colors are obtained by aging the fourth, fifth, and sixth percentages substantially the same amount of time; and creating a third column having color-filled boxes filled with the seventh color, eighth, and ninth colors. In this method, the first percentage may be substantially equal to the fourth percentage, the second percentage may be substantially equal to the fifth percentage, and the third percentage may be substantially equal to the sixth percentage. Additional dilutions and media may be used along the same lines as these steps to create additional columns and/or additional rows within each column.

The method also may comprise providing a gap between the first column and the second column. It may include providing indicators identifying each of the columns and each row across the columns. In addition, it may include providing a legend identifying values for the percentages and contents of the media.

In still another aspect of the invention, a method for evaluating a sample of blood content in one medium of a plurality of media may comprise: identifying the sample medium; selecting a column in a table having a plurality of columns, wherein the column includes a plurality of rows displaying colors, and wherein the colors visually represent actual dilutions of blood in the sample medium; visually comparing the sample to the selected column; and identifying a closest color match between the sample and one of the rows. One of the columns visually displays blood diluted in saline, a second of the columns visually displays blood diluted in urine, and a third of the columns visually displays blood diluted in urine and then aged. In addition, each of the columns may have a corresponding identifier, and each color within a column may have a second corresponding second identifier, such that the method further may comprise identifying an identifier and a second identifier corresponding to the closest color match.

These and other features and advantages are evident from the following description of the present invention, with reference to the accompanying drawings.

### BRIEF DESCRIPTION OF THE SEVERAL VIEWS OF THE DRAWINGS

FIG. 1 is a front view of a device for analyzing and assigning descriptive values to the amount of blood in a plurality of media for blood at a plurality of stages of time to evaluate the degree of hematuria or other internal bleeding in a patient.

FIG. 2 is a rear view of the device of FIG. 1.

### DETAILED DESCRIPTION

Patients receiving medical treatment sometimes require the use of catheters to remove and collect urine from the patients' bladder. If a patient is experiencing internal bleeding, this bleeding may be reflected in the exit port of the catheter, and the degree and age of bleeding may indicate to a health care provider that various actions may need to be taken to stabilize or treat the patient. At times, however, the provider may not physically be present to view the catheter and may have to rely on the subjective analysis of an intermediary to convey information about the color of the fluid in the discharge port.

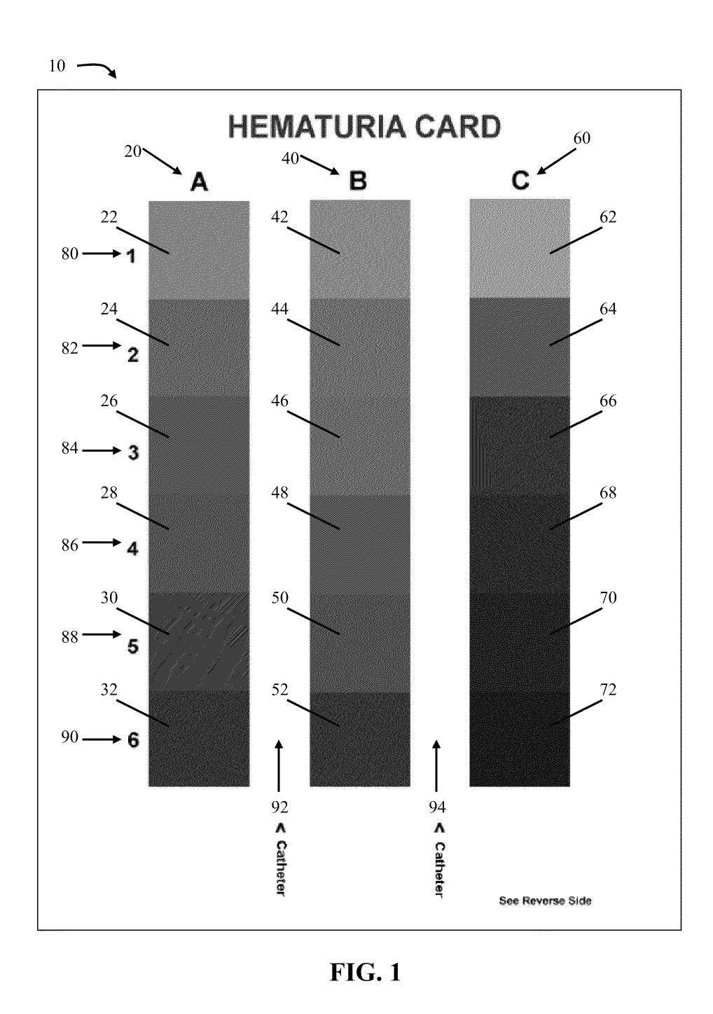

As seen in FIG. 1, in order to provide a qualitative analysis of the a device 10 for analyzing the degree of hematuria or other internal bleeding in a patient, which may comprise a display such as a card or computer screen having a plurality of columns 20, 40, 60 depicting a plurality of concentrations of blood in a plurality of media and, separately, at a plurality of times.

A plurality of columns, having column identifiers, may be provided, because the coloration produced by the presence of blood may vary, e.g., whether it is diluted in urine, saline, glycine, sorbitol, sterile water, or some other medium. In addition, blood hemolizes over time, i.e., hemoglobin in the blood breaks down. If the blood is older, whether hemolized or not, the coloration further may change, giving the liquid a brownish tint. Preferably, about three columns, 20, 40, 60 are provided. Column 20 may include various dilutions of blood in saline, column 40 may include various dilutions of blood in urine, and column 60 may include various dilutions of blood in urine that have been aged.

Each column may include a plurality of reference boxes, with each box representing an increase in blood concentration. Between about 3 and about 10 reference boxes may be provided for each column, preferably between about 5 and about 8 reference boxes, and in one embodiment, 6 reference boxes may be provided. Each column may include the same number of reference boxes as the other columns, although it is possible for each column to have more or fewer reference boxes than the other columns. In the embodiment shown in FIG. 1, column 20 may include reference boxes 22, 24, 26, 28, 30, and 32. Column 40 may include reference boxes 42, 44, 46, 48, 50, and 52. Column 60 may include reference boxes 62, 64, 66, 68, 70, and 72.

Reference boxes may be substantially similarly sized within each column and as between columns, which may create rows spanning columns, such as rows 80, 82, 84, 86, 88, and 90. In addition, the dilution percentages may be substantially equal across each row, which may provide an initial quantitative indicator of the degree of bleeding, although other tests may be used to confirm or definitively establish a quantitative result.

As mentioned above, a plurality of columns may be provided to correspond to a plurality of media in which the blood is diluted. For example, multi-port catheters may be used both to collect urine from the patient's bladder, and to irrigate the bladder, often with saline. These catheters may include, inter alia, an in port and an out port. During irrigation, saline (e.g., about 0.9% normal saline), glycine, sorbitol, or sterile water may be carried into the bladder through the in port and removed from the bladder via the out port. If no irrigation occurs, urine that is produced may comprise the fluid traveling through the out port. A plurality of columns allows a user to employ a single device 10 to examine the contents of the out port for the presence of blood, whether irrigation is being performed or not.

In addition, both active and past bleeding may result in hematuria, but “old” blood may appear visually different than a same percentage of “new” blood. Whether the blood is “old” or “new” may be important to the health care provider because “old” blood may indicate that bleeding has ceased.

The inclusion of “old” blood images also may be important because it may allow device 10 to be used at multiple times to determine qualitatively the progression of hematuria. For example, a user may compare the fluid in the out port of a catheter at time t0 and note the column and row indicators to which the fluid most closely relates. Then, the user may compare the fluid in the out port at a later time, e.g., t0+48 hours, note the column and row indicators to which that fluid most closely relates, and then compare the first and second notes for any differences. If, at time t0, an active bleed is indicated, column 40 may be used. Then, at time t0+48, the coloration of the fluid may suggest to the user to use column 40, which may indicate to the user that the bleed has not stopped, or it may suggest that column 60 should be used, which may indicate that bleeding has stopped. In the former case, a change to a lighter reference box may indicate that bleeding is slowing, while a change to a darker reference box may indicate that bleeding is worsening.

Alternatively, the individual that performs the t0+48 analysis may not be the same individual that performed the t0 analysis. However, the person that performed the t0 analysis may record the results of that analysis so that the individual performing the t+48 analysis may be able to assess more accurately the patient's progress.

It is preferable to compare the columns of device 10 to fluid in a catheter because this may provide the most accurate, real-time analysis of the degree of bleeding. Device 10 may include at least one, and preferably a plurality, of gaps 92, 94 between columns 20, 40, 60 and extending along the height of the columns. Tubes comprising catheter may be clear, which may allow for unaltered inspection of the contents of the catheter. Gaps 92, 94 may have a color that will not alter the user's perception of the color in the catheter, e.g., the columns may be white. In addition, gaps 92, 94 may have widths sized about as wide as or wider than the width of catheter out port tubing. This may allow the user to locate the catheter along gap 92, 94 for a side-by-side comparison of the catheter and the columns adjacent the gap, while preventing the coloring in each reference box from altering the user's perception of the color in the catheter. Device 10 may provide a visual aid to assist in providing a qualitative determination of the degree and age of bleeding that may be used by the user or conveyed to a health care provider, possibly not physically present, to determine what actions, if any, should be undertaken.

Colors in the reference boxes may be obtained from direct observation of actual samples. For example, with respect to column 20, boxes 22, 24, 26, 28, 30 and 32 may be obtained by creating samples by diluting increasing amounts of blood in saline (e.g., about 0.9% normal saline), glycine, sorbitol, or sterile water. The combination may be passed through a catheter, and the catheter and its contents may be photographed, imaged or otherwise have their color analyzed.

Boxes 22, 24, 26, 28, 30 and 32 then may be created by reproducing those images, e.g., by printing or displaying copies of the photographs or by printing or displaying solid color-blocks having RGB, CMYK, HSV, HSB, Hex Triplet, or other color quantification values substantially similar to those of the samples. As such, the colors shown in boxes 22, 24, 26, 28, 30, and 32 may represent actual blood dilutions viewed in the same manner the user may view the patient's sample, providing for better, more accurate analysis of the patient's blood content.

Substantially similar procedures may exist for creating boxes 42, 44, 46, 48, 50 and 52, and 62, 64, 66, 68, 70 and 72. In these cases, however, columns may be created by creating a combination of blood and urine that will be depicted in first reference block 42 in column 40. Then, that combination may be allowed to rest for a predetermined period of time, e.g., about 48 hours, after which it may be analyzed again, with its color depicted in first reference block 62 in column 60. A plurality of urine samples, prior to inclusion of blood, may have multiple different colors based on a variety of factors including, e.g., the producer's level of hydration. As such, reference boxes in columns 40 and 60 preferably may be obtained from the same initial urine sample so that the only variables affecting color preferably may be blood content and time.

Additional columns may be created by diluting blood in other media or aging blood for different lengths of time, and additional boxes may be created by diluting different percentages of blood in each selected medium.

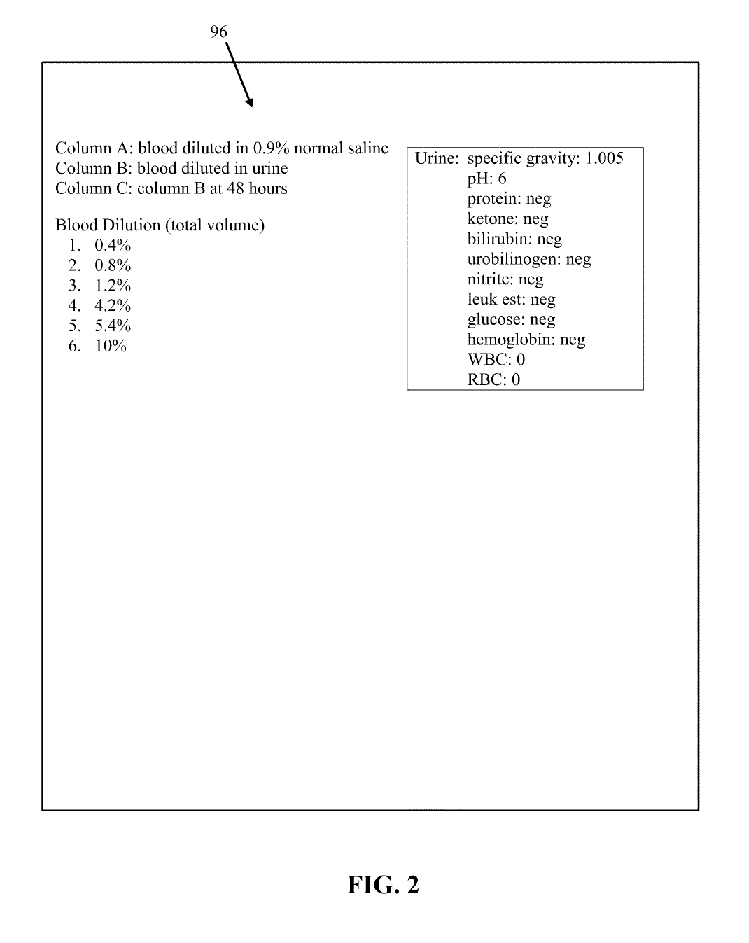

Device 10 may include a legend 94, e.g., on a side of device opposite columns 20, 40, 60 or on a separate portion of a display. Legend 94 may include explanations of the components of the images used to create the reference boxes. For example, legend 94 may describe the medium in which the blood is diluted and any time that elapsed between the dilution and when the color was analyzed. Legend 94 also may describe the relative concentrations of blood in each medium. If substantially similar concentrations are used across each row 80, 82, 84, 86, 88, 90, only one description may be necessary, as opposed to one for each column 20, 40, 60. As seen in FIG. 2, dilutions may be about 0.4%, about 0.8%, 1.2%, 4.2%, 5.4%, and about 10%, although other dilutions are possible.

In addition, legend 94 may include a description of the urine from which the samples were obtained. As seen in FIG. 2, urine preferably may be slightly acidic and negative for additional components. In one example, the urine used to produce the samples in columns 40, 60 may have the following characteristics: specific gravity: 1.005; pH: 6; negative for protein, ketone, bilirubin, urobilinogen, nitrites, leuk esterase, glucose, and hemoglobin; with white and red blood cell counts of 0. These values are exemplary and may be modified in other embodiments.

While the foregoing written description of the invention enables one of ordinary skill to make and use what is considered presently to be the best mode thereof, those of ordinary skill will understand and appreciate the existence of variations, combinations, and equivalents of the specific exemplary embodiment and method herein. The invention should therefore not be limited by the above described embodiment and method, but by all embodiments and methods within the scope and spirit of the invention as claimed.

What is claimed is:

1. A device for evaluating blood content in a plurality of media, comprising:
a plurality of columns, each column representing blood contents in different media;
a plurality of color-filled boxes in each of said plurality of columns, wherein, for each column, each color correlates to an actual dilution of blood in one of said different media.

2. A device according to claim 1, further comprising a gap between each of said plurality of columns, wherein said gap is at least as wide as tubing of a catheter containing a sample to be evaluated.

3. A device according to claim 1, wherein each of said plurality of columns has the same number of boxes, and further wherein said plurality of columns are substantially aligned so that said boxes form rows across said columns.

4. A device according to claim 3, further including indicators for each of said columns and each of said rows.

5. A device according to claim 3, wherein, for each row, boxes in said row have substantially the same blood dilution percentage.

6. A device according to claim 1, wherein one of said columns corresponds to blood diluted in saline, a second of said columns corresponds to blood diluted in urine, and a third of said columns corresponds to blood diluted in urine and then aged.

7. A method for making a device for evaluating blood content in a plurality of media, comprising:
diluting a first percentage of blood in a first medium to obtain a first color;
diluting a second percentage of blood in said first medium to obtain a second color;
diluting a third percentage of blood in said first medium to obtain a third color;
diluting a fourth percentage of blood in a second medium to obtain a fourth color;
diluting a fifth percentage of blood in said second medium to obtain a fifth color;
diluting a sixth percentage of blood in said second medium to obtain a sixth color;
creating a first column having color-filled boxes filled with said first color, said second color, and said third color; and
creating a second column having color-filled boxes filled with said fourth color, said fifth color, and said sixth color.

8. A method according to claim 7, further comprising:
diluting a seventh percentage of blood in a third medium to obtain a seventh color;
diluting an eighth percentage of blood in said third medium to obtain an eighth color;
diluting a ninth percentage of blood in said third medium to obtain a ninth color; and
creating a third column having color-filled boxes filled with said seventh color, said eighth color, and said ninth color.

9. A method according to claim 7, further comprising:
aging said fourth percentage of blood in said second medium to obtain a seventh color;
aging said fifth percentage of blood in said second medium to obtain an eighth color;
aging said sixth percentage of blood in said second medium to obtain a ninth color,
wherein said seventh, eighth, and ninth colors are obtained by aging said fourth, fifth, and sixth percentages substantially the same amount of time; and
creating a third column having color-filled boxes filled with said seventh color, said eighth color, and said ninth color.

10. A method according to claim 7, wherein said first percentage is substantially equal to said fourth percentage, said second percentage is substantially equal to said fifth percentage, and said third percentage is substantially equal to said sixth percentage.

11. A method according to claim 7, further comprising providing a gap between said first column and said second column.

12. A method according to claim 7, further comprising providing indicators identifying each of said columns and each row across said columns.

13. A method according to claim 7, further comprising providing a legend identifying values for said percentages and contents of said media.

14. A method for evaluating a sample of blood content in one medium of a plurality of media, comprising:
identifying said sample medium;
selecting a column in a table having a plurality of columns, wherein said column includes a plurality of rows displaying colors, and wherein said colors visually represent actual dilutions of blood in said sample medium;
visually comparing said sample to said selected column; and
identifying a closest color match between said sample and one of said rows.

15. A method according to claim 14, wherein said sample is within a catheter.

16. A method according to claim 14, wherein one of said columns visually displays blood diluted in saline, a second of said columns visually displays blood diluted in urine, and a third of said columns visually displays blood diluted in urine and then aged.

17. A method according to claim 14, wherein each of said columns has a corresponding identifier and each color within a column has a second corresponding second identifier, further comprising:
identifying an identifier and a second identifier corresponding to said closest color match.
